(19) United States
(12) Patent Application Publication
(10) Pub. No.: US 2025/0090077 A1
(43) Pub. Date: Mar. 20, 2025

(54) DYNAMIC ELECTROANATOMICAL MAPPING

(71) Applicant: Boston Scientific Scimed, Inc., Maple Grove, MN (US)

(72) Inventors: Allan Charles Shuros, St. Paul, MN (US); Matthew Ferrante, Cambridge, MA (US)

(21) Appl. No.: 18/889,192

(22) Filed: Sep. 18, 2024

Related U.S. Application Data
(60) Provisional application No. 63/583,833, filed on Sep. 19, 2023.

(51) Int. Cl. A61B 5/367; A61B 5/00; A61B 5/287; A61B 5/339; A61B 18/00; A61B 18/14; A61B 90/00

(52) U.S. Cl. CPC A61B 5/367; A61B 5/287; A61B 5/339; A61B 5/6852; A61B 18/1492; A61B 90/08; A61B 2018/00351; A61B 2018/00577; A61B 2018/00839; A61B 2090/0807; A61B 2560/0468

(57) ABSTRACT

A system to generate an electroanatomical map of patient's heart is disclosed. The system includes a catheter including an electrode to detect physiological signals from within the patient's heart and a location sensor to generate location signals representative of a location of the catheter within the patient's heart, a display device, and a controller. The controller determines a catheter context characteristic based on the location signals, collects the physiological signals according to a collection parameter. The collection parameter is based on the determined catheter context characteristic. The controller further collects anatomical location signals corresponding to a measurement location associated with each of the physiological signals and generates, on the display device, a three-dimensional representation of the patient's heart based on the physiological signals and the anatomical location signals.

### CROSS REFERENCE TO RELATED APPLICATIONS

The present application claims priority to U.S. Provisional Patent Application No. 63/583,833, filed Sep. 19, 2023, the entire disclosure of which is incorporated herein by reference.

### TECHNICAL FIELD

The present disclosure relates to medical systems and methods for mapping an anatomical space of the body. More specifically, the present disclosure relates to systems and methods for cardiac, electroanatomical mapping.

### BACKGROUND

Catheter ablation is a minimally invasive electrophysiological procedure to treat a variety of heart conditions such as supraventricular and ventricular arrhythmia. Such procedures can involve a visualization of the heart and heart activity. Electroanatomical mapping is a visualization technique that allows a clinician to accurately determine the location of an arrhythmia, define cardiac geometry in three dimensions, delineate areas of anatomic interest, and permits imaging of the catheter assembly for positioning and manipulation. For instance, electroanatomical mapping involves the mapping of electrical activity in the heart based on cardiac signals, such as at various locations on the endocardium surface, to identify the site of origin of the arrhythmia followed by a targeted ablation of the site.

To perform such cardiac mapping, a catheter with an electrode assembly at a distal tip of the catheter can be inserted into the patient's heart chamber. In some examples of mapping, physiological signals from electrical activity of the heart are acquired with electrodes after the tip is in stable and steady contact with the endocardium surface of a particular heart chamber. Alternatively, or additionally, signals can be detected by non-contact electrodes along with information on chamber anatomy and relative electrode location to provide physiological information regarding the endocardium of the heart chamber. Location and electrical activity are measured, such as sequentially on a point-by-point basis in some examples, at about fifty to several hundred points on the internal surface of the heart to construct an electroanatomical map of the heart. The generated electroanatomical map can serve as the basis to decide on a therapeutic course of action such as tissue ablation, to alter the propagation of electrical activity in the heart and to restore normal heart rhythm.

### SUMMARY

In Example 1, a system to generate an electroanatomical map of patient's heart. The system comprising: a catheter including an electrode configured to detect a plurality of physiological signals from within the patient's heart and a location sensor configured to generate a plurality of location signals representative of a location of the catheter within the patient's heart; a display device; and a controller configured to: determine a catheter context characteristic based on the plurality of location signals; collect the plurality of physiological signals according to a collection parameter, the collection parameter based on the determined catheter context characteristic; collect a plurality of anatomical location signals corresponding to a measurement location associated with each of the plurality of physiological signals; and generate, on the display device, a three-dimensional representation of the patient's heart based on the plurality of physiological signals and the plurality of anatomical location signals.

In Example 2, the system of Example 1, wherein the catheter context characteristic is associated with a velocity of the catheter determined using the plurality of location signals.

In Example 3, the system of Example 2 wherein the collection parameter includes collecting the physiological signals at a first sampling rate associated with a first context characteristic and at a second sampling rate associated with a second context characteristic.

In Example 4, the system of Example 3 wherein the controller configured to generate includes the controller configured to employ a process applying a higher degree of interpolation to the physiological signals collected at the first sampling rate and applying a lower degree of interpolation to the physiological signals collected at the second sampling rate.

In Example 5, the system of Example 3 wherein the controller configured to generate includes the controller configured to employ a process applying a higher degree of smoothing to the physiological signals collected at the first sampling rate and applying a lower degree of smoothing to the physiological signals collected at the second sampling rate.

In Example 6, the system of any of Examples 3-5, wherein the first sampling rate is applied when the first context characteristic is a velocity of from about 10 to about 50 mm/s and wherein the wherein the second sampling rate is applied when the second context characteristic is a velocity of from about 2 to about 10 mm/s.

In Example 7, the system of Example 1, wherein the catheter context characteristic is associated with a location of the catheter in the heart determined using the plurality of location signals.

In Example 8, the system of Example 1, wherein the electrode includes a mapping electrode and the location sensor includes a magnetic field sensor.

In Example 9, the system of any of Examples 1-8, wherein the mapping process includes a plurality of mapping processes, and each mapping process of the plurality of mapping processes includes an associated set of parameters.

In Example 10, the system of Example 9, wherein parameter values for each of the associated parameters are automatically adjusted based on the catheter context characteristics of the catheter.

In Example 11, the system of any of Examples 1-10, wherein the controller is configured to adjust the set of values via a lookup table.

In Example 12, the system of any of Examples 1-11, including a graphical display wherein the controller is configured to generate a visualization of the anatomy on the graphical display.

In Example 13, the system of any of Examples 1-12, wherein adjusting the set of values includes calculating the set of values based on the catheter context characteristics.

In Example 14, the system of any of Examples 1-13, wherein the catheter includes mapping and ablation electrodes.

In Example 15, the system of any of Examples 1-13, wherein the catheter includes mapping and ablation electrodes.

In Example 16, a system to generate an electroanatomical map of patient's heart. The system comprising: a catheter including an electrode configured to detect a plurality of physiological signals from within the patient's heart and a location sensor configured to generate a plurality of location s456-cf12-.ignals representative of a location of the catheter within the patient's heart; a display device; and a controller configured to: determine a catheter context characteristic based on the plurality of location signals; collect the plurality of physiological signals according to a collection parameter, the collection parameter based on the determined catheter context characteristic; collect a plurality of anatomical location signals corresponding to a measurement location associated with each of the plurality of physiological signals; and generate, on the display device, a three-dimensional representation of the patient's heart based on the plurality of physiological signals and the plurality of anatomical location signals.

In Example 17, the system of Example 16, wherein the catheter context characteristic is associated with a velocity of the catheter determined using the plurality of location signals.

In Example 18, the system of Example 17 wherein the collection parameter includes collecting the physiological signals at a first sampling rate associated with a first context characteristic and at a second sampling rate associated with a second context characteristic.

In Example 19, the system of Example 18 wherein the controller configured to generate includes the controller configured to employ a process applying a higher degree of interpolation to the physiological signals collected at the first sampling rate and applying a lower degree of interpolation to the physiological signals collected at the second sampling rate.

In Example 20, the system of Example 18 wherein the controller configured to generate includes the controller configured to employ a process applying a higher degree of smoothing to the physiological signals collected at the first sampling rate and applying a lower degree of smoothing to the physiological signals collected at the second sampling rate.

In Example 21, the system of Example 18, wherein the first sampling rate is applied when the first context characteristic is a velocity of from about 10 to about 50 mm/s and wherein the wherein the second sampling rate is applied when the second context characteristic is a velocity of from about 2 to about 10 mm/s.

In Example 22, the system of Example 16, wherein the mapping process includes a plurality of mapping processes, and each mapping process of the plurality of mapping processes includes an associated set of parameters.

In Example 23, the system of Example 16, wherein the parameter is associated with a parameter value, and the parameter value for the parameter is adjusted based on the catheter context characteristic.

In Example 24, the system of Example 23, wherein the controller is configured to adjust the parameter value via a lookup table in a memory device.

In Example 25, the system of Example 23, wherein the controller is configured to adjust the parameter value via a calculation.

In Example 26, the system of Example 16, wherein the catheter includes mapping and ablation electrodes.

In Example 27, a method for generating an electroanatomical map of a patient's heart, the method comprising: receiving a plurality of physiological signals from a catheter within the patient's heart and a plurality of location signals representative of a location of the catheter within the patient's heart; determining a catheter context characteristic based on the plurality of location signals; collecting the plurality of physiological signals according to a collection parameter, the collection parameter based on the determined catheter context characteristic; collecting a plurality of anatomical location signals corresponding to a measurement location associated with each of the plurality of physiological signals; and generating, on the display device, a three-dimensional representation of the patient's heart based on the plurality of physiological signals and the plurality of anatomical location signals.

In Example 28, the method of Example 27, wherein the catheter context characteristic is associated with a velocity of the catheter determined using the plurality of location signals.

In Example 29, the method of Example 28, wherein the collecting the plurality of physiological signals according to a collection parameter includes collecting the physiological signals at a first sampling rate associated with a first context characteristic and at a second sampling rate associated with a second context characteristic.

In Example 30, the method of Example 29, wherein the generating includes applying a higher degree of interpolation to the physiological signals collected at the first sampling rate and applying a lower degree of interpolation to the physiological signals collected at the second sampling rate.

In Example 31, the method of Example 28, wherein the generating includes applying a higher degree of smoothing to the physiological signals collected at the first sampling rate and applying a lower degree of smoothing to the physiological signals collected at the second sampling rate.

In Example 32, method of Example 27, wherein the parameter is associated with a parameter value, and further comprising adjusting the parameter value based on the catheter context characteristic.

In Example 33, a system to generate an electroanatomical map of a patient's heart, the system comprising: a catheter including an electrode configured to detect a plurality of physiological signals from within the patient's heart and a location sensor configured to generate a plurality of location signals representative of a location of the catheter within the patient's heart; a display device; and a controller configured to: determine a catheter context characteristic based on the plurality of location signals; collect the plurality of physiological signals according to a selected value of a collection parameter from a range of values, the selected value based on the determined catheter context characteristic; collect a plurality of anatomical location signals corresponding to a measurement location associated with each of the plurality of physiological signals; and generate, on the display device, a three-dimensional representation of the patient's heart based on the plurality of physiological signals and the plurality of anatomical location signals.

In Example 34, the system of Example 33, wherein the controller is configured to select the value via a lookup table.

In Example 35, the system of Example 34, wherein the controller is configured to input a velocity of the catheter into the lookup table to obtain the selected value.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an exemplary clinical setting for treating a patient, and for mapping a heart of the patient, using an electrophysiology system, in accordance with embodiments of the subject matter of the disclosure.

FIG. 2 is a block diagram illustrating an example system for use with the example electrophysiology system of FIG. 1.

FIG. 3 is a block diagram illustrating an example controller for use with the example system of FIG. 2.

FIG. 4 is a flow diagram illustrating an example configuration of the example controller of FIG. 3.

FIGS. 5A and 5B are diagrams illustrating example visualizations generated with the example controller of FIG. 4.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

Electroanatomical mapping programs generate three-dimensional representations of the anatomy as a three-dimensional mesh, or mesh. The mesh is created via a suite of mapping processes in the electroanatomical program that apply on a number of parameters. Some of the parameter values can be user-modifiable during a mapping procedure, such as parameter values related to tightness and to beat gating, while other parameter values are generally preselected and not user-modifiable, such as parameter values related to smoothing and coarsening. Parameter values for the mapping processes are typically selected based on various assumptions related to the anatomy to be mapped, catheter geometry, and catheter movement, and are typically fixed during the data acquisition. Default parameter values are typically chosen as a compromise between mapping accuracy and speed of procedure. Such default parameter values, however, can be suboptimal for many circumstances of data collection in mapping procedures, and are often dependent on characteristic of how a clinician collects the data with the mapping catheter. For example, the use of beat acceptance criteria or other parameter values in generating anatomical representations can be too stringent in some areas of the heart, such as much of the heart, which impedes rapid data collection, and other areas of the anatomy benefit from detailed data collection, such as the pulmonary veins, arrhythmogenic circuits and breakthroughs, and the left atrial appendage. Clinicians tend to adjust their catheter maneuvering techniques based on whether a detailed anatomical map will be helpful. For example, a clinician can rapidly maneuver a catheter over areas of the heart to gather coarse anatomy and deliberately maneuver the catheter over areas of greater interest.

The disclosure provides mapping processes in an electroanatomical mapping program to generate three-dimensional representation of anatomy that are dynamically adjusted based on determined catheter context characteristics. Examples of determined catheter context characteristics include position of the catheter, velocity of the catheter, orientation of the catheter, whether the catheter is in contact with a wall of the anatomy, or the location of the anatomy in which data is being collected.

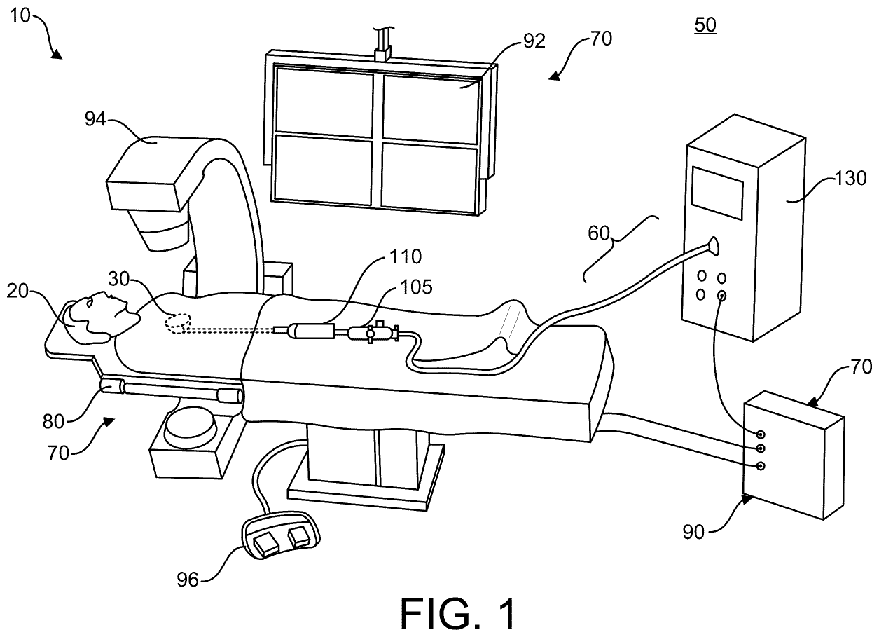

FIG. 1 illustrates an example clinical setting 10 for treating a patient 20, which can include mapping a heart 30 of the patient 20, using an electrophysiology system 50, in accordance with the disclosure. The electrophysiology system 50 includes a catheter system 60 and an electroanatomical mapping (EAM) system 70. The example catheter system 60 includes a catheter 105 to obtain electrical signals of the heart, an introducer sheath 110, and can include a console 130 to receive the electrical signals obtained from the heart, such as in the case of a mapping catheter. In some examples, the console can also provide ablation energy such as in the case of the catheter 105 configured as a mapping and ablation catheter. Additionally, the catheter system 60 includes various connecting elements, such as cables, that operably connect the components of the catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The introducer sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter 105 includes an elongated catheter shaft and distal end region configured to be deployed proximate target tissue, such as within a chamber of the patient's heart. The shaft can extend from an access point in the patient to the target tissue and generally defines a longitudinal axis of the catheter 105. The distal end region may include a basket, balloon, spline, configured tip, or other electrode deployment mechanism coupled to the shaft. The electrode deployment mechanism includes an electrode assembly, or array, comprising an electrode to obtain electrical signals from the heart. For example, the electrode assembly can include a plurality of spaced-apart electrodes or multiple spaced-apart sets or groups of spaced-apart electrodes. For instance, the electrode deployment mechanism includes a plurality of splines configured to form a basket, and at least a some of the electrodes are disposed on the splines.

The catheter 105 is configurable in a plurality of states. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded state.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the catheter 105, such as a plug configured to be mechanically and electrically coupled to the console 130, for example, either directly or via intermediary electrical conductors such as cabling.

The electrodes are configured for, among other things, sensing cardiac electrical signals, localization of the electrode assembly within the patient anatomy such as via the EAM system 70, and to determine proximity to target tissue within the anatomy. In some examples, the catheter 105 can be a mapping and ablation catheter, and the electrodes can include ablation electrodes that are configured to deliver ablation electric field energy and sensing electrodes, or mapping electrodes, for mapping purposes. The ablation electrodes are configured to receive pulsed electrical signals or waveforms from the console 130 and, in the example of electroporation ablation, create pulsed electric fields sufficient to ablate target tissue via irreversible electroporation. The mapping electrodes in the electrode assembly can be electrically coupled to a one or more lead conductors that extends the length of the shaft that are configured to carry an electrical signal received at the mapping electrode. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

Mapping electrodes on the catheter 105 can measure physiological (electrical) signals and generate electrical output signals that can be processed by the mapping and navigation controller 90 to generate an electroanatomical map. In some instances, electroanatomical maps are generated before ablation for determining the electrical activity of the cardiac tissue within a chamber of interest. In some instances, electroanatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The electroanatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping.

The EAM system 70 is configured to generate the electroanatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices, such as in addition to or instead of magnetic tracking. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the catheter 105 or another catheter or probe equipped with sensing electrodes, to generate, and display via the display 92, detailed three-dimensional geometric anatomical maps or representations of the heart tissue and voids such as cardiac chambers as well as electroanatomical maps in which cardiac electrical activity of interest is superimposed on the geometric anatomical maps.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

Each cardiac electrical signal may include a number of intracardiac electrograms (EGMs) sensed within a patient's heart and may include any number of features that may be ascertained by aspects of the system 50. Examples of cardiac electrical signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac electrical signal feature can refer to one or more features extracted from one or more cardiac electrical signals, derived from one or more features that are extracted from one or more cardiac electrical signals. Additionally, a representation, on a cardiac or a surface map, of a cardiac electrical signal feature may represent one or more cardiac electrical signal features, an interpolation of a number of cardiac electrical signal features. Each cardiac signal also can be associated with a set of respective position coordinates that corresponds to the location at which the cardiac electrical signal was sensed. Each of the respective position coordinates for the sensed cardiac signals can include three-dimensional Cartesian coordinates, polar coordinates, or another coordinate system. The cardiac signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the catheter 105 is displaced to multiple locations within the heart chamber into which the catheter 105 is inserted. At each location to which the catheter 105 is moved, the electrodes and sensors acquire physiological signals resulting from the electrical activity in the heart along with positional, or spatial, information of the catheter 105. The spatial information is used in building a three-dimensional grid of the anatomy during mapping. To perform a mapping procedure and reconstruct physiological information on the endocardium surface, the EAM system 70 may align a coordinate system of the catheter 105 with the endocardium surface's coordinate system, or vice versa. Alternatively, or additionally, the grid may be used to capture EGMs, and select mapping values based on statistical distributions associated with nodes of the grid. The EAM system 70 also can perform post-processing operations on the physiological information to extract and display useful features of the information to the operator of the system 50.

In generating an example electroanatomical map, a data stream including multiple signals, including signals received from the mapping electrodes of the catheter 105, is input into the EAM system 70. During the automated electroanatomical mapping process, the data stream provides a collection of physiological and location signals that serve as an input to the mapping process. The signals may be collected directly by the mapping system, obtained from another system using an analog or digital interface, or both. The data stream 302 can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

A surface geometry construction process generates the anatomical surface, such as a three-dimensional representation of the patient's anatomy, on which the electroanatomical map is displayed. Surface geometry can be constructed, for example, using aspects of a system as described in U.S. Pat. No. 8,103,338 to Doron Harlev et al., titled “IMPEDANCE BASED ANATOMY GENERATION,” issued on Jan. 24, 2012; or U.S. Pat. No. 8,948,837, to Doron Harlev et al., titled “ELECTROANATOMICAL MAPPING,” issued on Feb. 3, 2015, the contents of each of which are incorporated by reference herein. Additionally, or alternatively, an anatomical shell can be constructed by the EAM system 70 by fitting a surface on electrode locations that are determined either by the user or automatically to be on the surface of the chamber. In addition, a surface can be fit on the outermost electrode or catheter locations within the chamber. Surface geometry can be represented as a mesh including a collection of vertices, or points, and connectivity between them to form, for example, triangles. Alternatively, surface geometry can be represented by different functions such as higher order meshes, non-uniform rational basis splines (NURBS), or curvilinear shapes.

The surface map data may provide information on cardiac electrical excitation, cardiac motion, tissue proximity information, tissue impedance information, force information, and other collected information desirable to the clinician. The combination of map dataset and surface geometry data allows for surface map generation. The surface map is a collection of values or waveforms, such as EGMs, on the surface of the chamber of interest, and the map dataset can contain data that is not on the cardiac surface. Displayed maps can be computed and displayed separately or overlaid on top of each other. Additional details of electroanatomical mapping processes and related systems are described in U.S. Pat. No. 11,272,887 to Brian Stewart, et al., titled “ELECTROANATOMICAL MAPPING TOOLS FACILITATED BY ACTIVATION WAVEFORMS,” issued on Mar. 15, 2022, the contents of which are incorporated by reference herein.

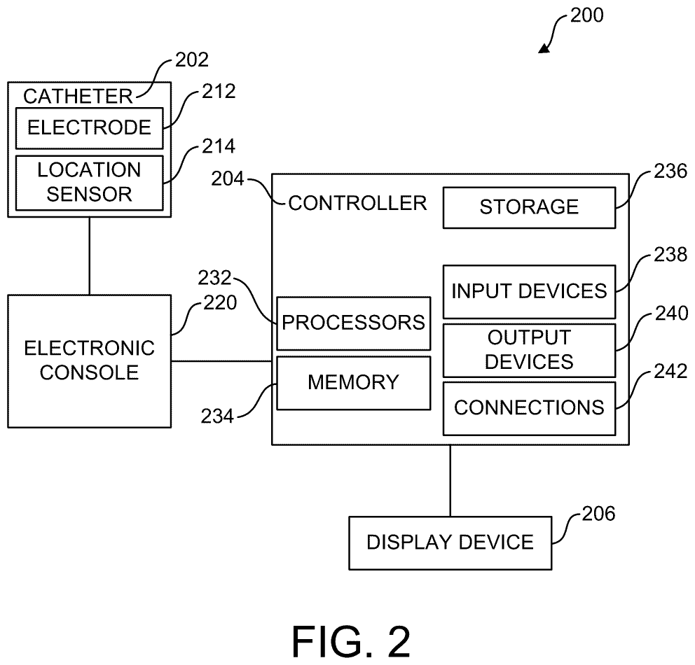

FIG. 2 illustrates an example system or sub-system 200 that can be used the example electrophysiology system 50 of FIG. 1, such as including the EAM system or mapping and navigation controller 90 to generate an electroanatomical map of a patient's heart. The system 200 includes a catheter 202, controller 204, and display device 206. The catheter 202 is coupled to a controller 204, and the controller 204 is coupled to a display device 206. The catheter 202 includes an electrode 212 configured to detect a plurality of physiological signals from within the patient's heart and a location sensor 214 configured to generate a plurality of location signals representative of a location of the catheter within the patient's heart. In embodiments, the catheter 202 is implemented as catheter 105 and can include a plurality of mapping electrodes. In the illustrated example, the catheter 200 is coupled to an electrical or electronic console 220, such as console 130, to receive, process, and provide physiological signals and location signals from the catheter 202 to the controller 204. The controller 204 is configured to receive the provided physiological signals and location signals from the catheter 202 and to generate a three-dimensional representation of the patient's heart, such as on the display 206, based on the plurality of physiological signals and the plurality of anatomical location signals.

In embodiments, the controller 204 is employed in the clinical environment and used to host or run a computer application included on one or more computer readable storage mediums storing computer executable instructions for controlling the controller 204, such as a computing device, to perform a process. For example, the controller is configured to run an electroanatomical mapping program constructed in accordance with the disclosure. The controller 204 can take one or more of several forms. Such forms include a tablet, a personal computer, a workstation, a server, a handheld device, a dedicated electronic device and can be a stand-alone device or configured as part of a computer network. In a basic hardware configuration, controller 204 typically includes a processor system having one or more processing units, i.e., processors 232, and memory 234. By way of example, the processing units may include two or more processing cores on a chip or two or more processor chips. In some embodiments, the controller 202 also includes one or more additional processing or specialized processors (not shown), such as a graphics processor for general-purpose computing on graphics processor units, to perform processing functions offloaded from the processor 232. The memory 234 may be arranged in a hierarchy and may include one or more levels of cache. Depending on the configuration and type of computing device, memory 104 may be volatile (such as random access memory (RAM)), non-volatile (such as read only memory (ROM), flash memory, etc.), or some combination of the two.

Controller 204 can also have additional features or functionality. For example, controller 204 includes additional storage 236. Such storage 236 may be removable or non-removable and can include magnetic or optical disks, solid-state memory, or flash storage devices. Computer storage media includes volatile and nonvolatile, removable and non-removable media implemented in any suitable method or technology for storage of information such as computer readable instructions, data structures, program modules or other data. Memory 234 and storage 236 such as removable storage and non-removable storage are all examples of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, universal serial bus (USB) flash drive, flash memory card, or other flash storage devices, or any other storage medium that can be used to store the desired information and that can be accessed by controller 204. Accordingly, a propagating signal by itself does not qualify as storage media. Any such computer storage media may be part of controller 204.

In embodiments, controller 204 includes input devices 238, output devices 240, and connections 242. In some embodiments input devices and output device can be peripheral device coupled to the controller 204 via connections 242. Controller 204 can include one or more connections 242, such as USB connections, display ports, proprietary connections, and others to connect to various devices to provide inputs and outputs to the controller 204. Examples of input devices 238 include keyboard, pointing device (e.g., mouse, track pad), stylus, voice input device, and a touch input device (e.g., touchscreen). Examples of output devices 240 may include devices such as a lights, screens, display 206, speakers, and a printer. Connections 242 also include communication connections that allow controller 204 to communicate with other computers/applications. Example communication connections can include an Ethernet interface, a wireless interface, a bus interface, a storage area network interface, and a proprietary interface. The communication connections can be used to couple the controller 204 to a computer network, which can be classified according to a wide variety of characteristics such as topology, connection method, and scale. A network is a collection of computing devices and possibly other devices interconnected by communications channels that facilitate communications and allows sharing of resources and information among interconnected devices. Examples of computer networks include a local area network, a wide area network, the internet, or other network.

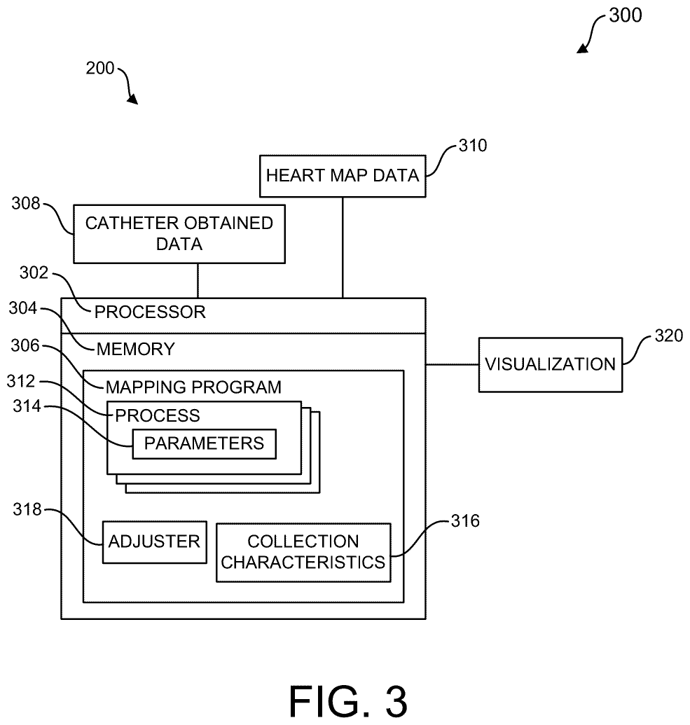

FIG. 3 illustrates an example controller 300 that can be used with the example electrophysiology system 50, such as a controller of the example EAM system 70 or the mapping and navigation controller 90 and as illustrated as controller 206 in system 200. The controller 300 can be implemented to provide an electroanatomical map of the heart based on characteristics of the catheter during data collection. The controller 300 can include a processor 302 and a memory 304. The memory 304 stores processor executable instructions 306. In one example, the processor executable instructions 306 are in the form of a program, such as a computer program or application. The processor 302 can execute the instructions 306 that can be included in configuring the controller 300. In one example, the controller 300 can be implemented to include a computing device such as a laptop computer, a workstation, a desktop computer, a tablet, or a smartphone. In such examples, the controller 300 can include additional components or devices such as a display, a touchscreen, speakers or other output devices, a keyboard or other input devices, or communication circuitry such as computer network adapters. The controller 200 may be implemented in a variety of architectures and components, such as the processor 302 and memory 304, may be distributed in various locations.

In one example, the processor 302 may include a plurality of main processing cores to run an operating system and perform general-purpose tasks on an integrated circuit. The processor 302 may also include built-in logic or a programmable functional unit, also on the same integrated circuit. In additional to multiple general-purpose, main processing cores and the application processing unit, controller 300 can include other devices or circuits such as graphics processing units or neural network processing units with the main processing cores. For example, the controller 300 may be used to perform other tasks.

Memory 304 is an example of computer storage media. Memory 304 is a non-transitory, processor readable memory device. Accordingly, a propagating signal by itself does not qualify as storage media or memory 304.

The controller 300 may be configured to receive inputs or information from the electrophysiology system 50, such as inputs from the electroporation catheter system 60 and EAM system 70 including the electroporation console 130 and the mapping and navigation controller 90, for storage in memory 204 and use by the instructions 206. For example, the controller receives inputs representative of data obtained with the catheter system 60, such as data determined from electrical signals received from the electrodes 210, such as mapping electrodes, and tracking devices 212, or catheter obtained data 308. Catheter obtained data 308 can include data determined from electrical signals such as EGMs, ECGs, electrode location information, tissue proximity information, force or contact information, catheter tip or tissue temperature, acoustic information, catheter electrical coupling information, catheter deployment shape information, electrode properties, respiration phase, blood pressure, other physiological information. In some examples, catheter obtained data can include or be supplemented with other information collected from sensors during mapping of the heart. In some examples, the controller 300 can receive an input representative of the anatomical map of the heart, or previously collected heart map data 310, which previously collected heart map data 310 can include the data regarding representations of the geometric anatomical map of the heart and the electro-anatomical map of the heart, such as from the EAM system 70. Previously collected heart map data can include data previously collected, such as in the same procedure using a different map dataset, or with a different modality such as CT, MRI, ultrasound, or rotational angiography, and registered to the catheter locating system.

The computer program 306 can be an electroanatomical mapping program to generate an electroanatomical map of a patient's heart. One aspect of the electroanatomical mapping program 306 includes a mapping process, or a suite of mapping processes 312 that impact anatomy generation, or the creation of three-dimensional representations of the anatomy, such as the patient's heart. For example, the suite of mapping processes 312 can include one or more surface geometry construction process that generates the anatomical surface, such as a three-dimensional representation of the patient's anatomy, on which the electroanatomical map is displayed. In one example, a surface geometry process of the suite of mapping processes 312 can create and represent the heart as a mesh including a collection of vertices, or points, and connectivity between them to form, for example, triangles. The mapping process, or suite of mapping processes 312 can each include a plurality of parameters 314 as variables. The parameters 314 can be assigned parameter values in the instructions. In some cases, the parameter values are user selectable, such in parameters related to tightness and beat gating, and can be input or modified by a user at a prompt in an input interface of the program 306. In other cases, the parameter values are not user-modifiable via an input interface such as some parameters related to smoothing or coarsening.

An aspect of the electroanatomical mapping program 306 includes context characteristics 316. Context characteristics data 316 includes information about the context of the catheter in the collection of the catheter obtained data 308. For example, context characteristics data can include characteristics such as physical movement of the catheter in obtaining the data. Physical movement can include velocity, orientation of the catheter, vector of travel, and path of the catheter movement in obtaining the data. Other context characteristics can include whether the catheter contacts a wall of the heart or anatomy, or region or structure of the heart from which data is collected. Still other context characteristics can include beat acceptance criteria and whether the beat is accepted, presence of ectopy/arrhythmia, phases of the respiration cycle, and phases of the cardiac cycle. Context characteristics data 316 can be determined from catheter obtained data 308, such as location information and time. In one example, context characteristics can be measured with reference to another catheter near the therapy site. The program 306 also includes an adjuster 318, which can be used to adjust parameter values, such as for user selectable parameter values as well not user-modifiable parameters, based on context characteristics 316. The adjuster 318 automatically and dynamically adjusts the parameter values of the parameters 314 based on context characteristics, such as velocity of the catheter, as based on context characteristics data 316.

The controller 300 via program 306 is configured to generate a visualization 320 of the electroanatomical map of patient's heart on a graphical display, such as display 92 or display device 206.

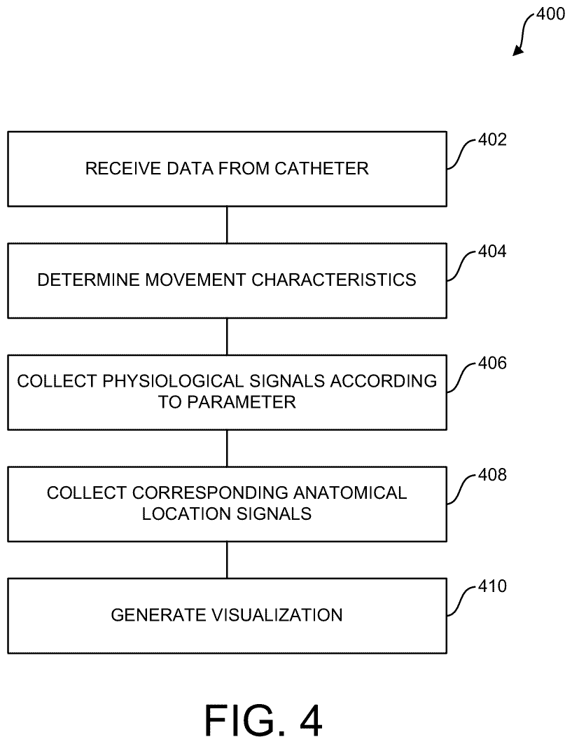

FIG. 4 illustrates a process 400 of configuring a controller, such as controller 300, to generate an electroanatomical map of a patient's heart. In one example, the controller is implemented as part of the EAM system 70 and operably coupled to the electroporation catheter system 60. Process 400 includes receiving data from catheter system such as a mapping catheter, or a catheter with mapping features, at 402. In a particular example, the data from catheter can be in the form of electrical signals received from an electrode, such as a plurality of mapping electrodes in the electrode assembly of the catheter and from a location sensor, such as a magnetic field sensor associated with a magnetic tracking system or EAM electrodes to detect an electrical field associated with an impedance tracking system, and obtained from a mapping procedure from within the patient's heart. In one example of process 400, catheter obtained data 308 is received at 402. Catheter obtained data 308 can include data from a plurality of physiological signals obtained from within the patient's heart detected with an electrode, such as a mapping electrode, and a plurality of location signals representative of a location of the catheter within the patient's heart obtained with a location sensor.

Context characteristics, such as context characteristics data 316, are determined based on the plurality of received location signals at 404. The electroanatomical mapping program 306 includes a mapping process related to the generation of a three-dimensional representation of anatomy in the electroanatomical map. The mapping process includes a set of parameters is applied to the received data at 402. In some embodiments, a set of values for the parameters are adjusted based on the context characteristics of the catheter. For example, the set of values are automatically determined based on the context characteristics, and the determined set of values are applied in the mapping process to generate the anatomy. The context characteristics are determined dynamically and automatically, such as continuously or at a sampling rate. Similarly, the parameter values are adjusted dynamically and automatically based on the context characteristics, such as continuously or at a sampling rate.

A plurality of physiological signals are collected according to a collection parameter, the collection parameter based on the determined catheter context characteristic at 406. Also, a plurality of anatomical location signals corresponding to a measurement location associated with each of the plurality of physiological signals are collected at 408. For example, each physiological signal collected according to the catheter context characteristic at 406 is associated with an anatomical local signal corresponding to the measurement location of the physiological location. In one embodiment, the data collected based on the determine context characteristic are stored in memory as physiological information, location information pairs. A visualization of the electroanatomical map including the three-dimensional representation of the anatomy is generated at 410.

In one embodiment of process 400, the electrode assembly of the catheter is maneuvered proximate an anatomical target of interest by a clinician or operator, and the mapping electrodes and location sensors, and other sensors gather data at 402 in the form of electrical signals that can be processed and determined to be catheter context characteristics at 404. Catheter context characteristics can be related to the orientation of the electrode assembly, position of the catheter, whether the catheter is in contact with a wall of the anatomy, the location of the catheter within the anatomy, such as in which region or structure of the heart or body the electrode assembly is located, or context characteristics of the catheter, such as the velocity of the catheter or electrode assembly or whether the catheter is accelerating or decelerating and other information. In one example, velocity can be determined by distance per unit of time, such as millimeters per second, or can be determined by distance per beat of the heart, or distance per selected number of beats. Other measurements of velocity of motion are contemplated, such as number of grid cells in the three-dimensional representation of the heart per beat, or average velocity measurements.

The generation of a three-dimensional representation of anatomy in creating an electroanatomical map includes a mapping process, and the mapping process includes a set of parameters at 306. The generation of a three-dimensional representation of anatomy can include a plurality of mapping processes, and each mapping processes is associated with a set of parameters. The parameter values applied to the associated set of parameters are adjusted based on the catheter context characteristics as included in 404.

Determined catheter context characteristics can be related to the velocity of the electrode assembly as the catheter is maneuvered in the anatomy. For instance, a clinician may increase the speed of the catheter in areas to intended to provide coarse readings and decrease the speed of the catheter in areas intended to provide more detailed anatomy. If the catheter is maneuvered relatively quickly, or with relative greater velocity, parameter values can be adjusted to provide for more interpolation between data points, lower sampling rate for electrical signals, and apply continuous anatomy collection instead of beat-gated anatomy collection. If the catheter is maneuvered relatively slowly, or with relative less velocity, parameter values can be adjusted to provide for less interpolation between data points, greater sampling rate for electrical signals, and apply beat-gated anatomy collection instead of continuous anatomy collection. In one example, a range of high velocity catheter movement can include 10-50 millimeters per second and a range of low velocity catheter movement is 2-7 millimeters per second. Catheter movement above or below a range can trigger an alert to the clinician via the display device or a speaker to change the speed of the catheter to collect data more efficiently. In one example, an amount of interpolation or sampling rate is fixed for velocities above and below a selected range, and the amount of interpolation is a linear or geometrical function of the velocity within the selected range. In some embodiments, the range can change, or ranges can be sliding scale, depending on the circumstances of the anatomy collection. In some examples, grid resolution is adjusted in the same way as mesh tightness, i.e., based on the speed of the catheter, such that a higher resolution (smaller grid cells) is generated in response to slower speeds of the catheter. In still another example, velocity of the catheter can influence the strictness of anatomy generation. For instance, faster motion of the catheter can be interpreted by the controller as an indication of less need for detail. In this instance, beat acceptance restrictions can be removed or reduced and the amount of interpolation can be increased as a function of speed.

In another example, the context characteristics are based on a particular location of the electrode in a selected region of the anatomy. Parameter values can be selected based on the region of the anatomy. For instance, parameter values can be adjusted for selected locations in the anatomy, such as the pulmonary veins and left atrial appendage, to provide for anatomical accuracy relative to other parts of the anatomy. If the location data determined from the location sensor indicates the catheter is in a location significant to a particular electrophysiological procedure, such as the pulmonary veins, the location data can be applied to adjust parameters. In one embodiment, the location data can be associated with a location identifier, such as a broad range identifier like the pulmonary veins or right atrium. Based on the electrophysiological procedure, the location identifier can be used to adjust the parameter values. The catheter collecting data in the pulmonary veins for the procedure can yield parameter values that trigger continuous anatomy collection rather than beat gated collection and may include a higher sampling rate. The catheter collecting data in the right atrium for the procedure can yield parameter values that trigger beat-gated anatomy collection and may include a lower sampling rate.

The application of multiple context characteristics can affect parameter values in circumstances, as well. For instance, additional context characteristics received beyond just the speed of the catheter, such as its current state of contact with the endocardium, can result in an override of speed-based parameters settings and result in the application of stricter settings that will preserve the detail in circumstances when the catheter is in contact with the chamber wall. Additionally, the process 400 can apply or use information regarding the neighboring regions of the mesh to influence the tightness at a given location, as nearby locations in the mesh likely share similar tightness parameter values.

Parameter values based on catheter context characteristics can be determined in a variety of processes. In one example, parameter values for an associated mapping process can be determined via a look up table, such as a look up table stored in the memory 204. A given context characteristic or set of context characteristics are input into a look up table, and a set of parameter values to be applied to the parameters of the mapping process are provided. In another example, the parameter values may be determined by calculation. In still another example, the parameter values may be determined by interpolation, or calculation if the inputs are between nodes of a look up table. Parameter values can include data values of appropriate types, such as integers, floating point, strings, or Boolean, such as whether to apply to continuous or beat-gated collection, such as beatGated:=True. Parameter values can be adjusted for mapping processes related to generating three-dimensional representations of anatomy relative to parameter values for other processes of electroanatomical mapping. For instance, a clinician may maneuver the catheter too quickly for a process to accept a beat, but the speed of the catheter may not prevent other types of data collection particularly if the catheter is also in contact with the wall. For example, catheter context characteristics of speed and wall contact, as determined by local impedance of an electrode, for instance, can be suited for data collection related to anatomy via adjusted parameters, when beat information is rejected. In some embodiments, beat acceptance criteria is incorporated during the higher velocity data acquisition periods, such as quiescent periods of breathing (transthoracic impedance), quiescent periods of heart movement, and cycle length (such as to eliminate or remove ectopy/arrhythmias).

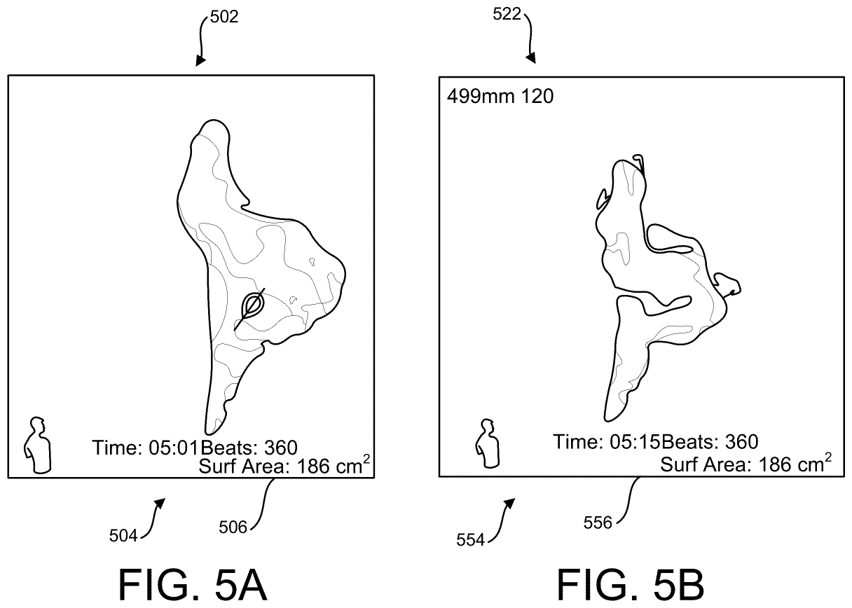

FIGS. 5A and 5B illustrate a comparison of visualizations generated by electroanatomical mapping processes. FIG. 5A illustrates a visualization 502 of a representation of a portion of patient's heart 504 on a display device generated from an electroanatomical mapping process of the prior art. In the process of the prior art, context characteristics are not based on how the catheter is maneuvered or on the on the plurality of location signals and parameter values associated with data interpolation are not dynamically adjusted. For instance, the parameter value associated with data interpolation is a default setting value and remains at the default setting for the entire period that physiological signals are collected for the represented portion of the patient's heart 504. The representation of the portion of the patient's heart 504 appears to include a relatively smooth surface 506. To a user with detailed knowledge of the anatomy and the mapping process, the representation of the portion of the patient's heart 504 appears to lack surface detail due to a webbing effect.

FIG. 5B illustrates a visualization 552 of a representation of a portion of the patient's heart 554 on a display device generated from electroanatomical mapping process 400. The portion of the heart in representation 554 is the same portion of the heart in representation 504. In embodiments of process 400, context characteristics are based on how the catheter is maneuvered or on the on the plurality of location signals and parameter values associated with data interpolation are automatically and dynamically adjusted. For instance, the parameter value associated with data interpolation is dynamically adjusted to values from a set of available values during the period that physiological signals are collected for the represented portion of the patient's heart 554. The representation 554 includes greater surface detail 556 in accordance with the actual surface of the patient's heart. The dynamically adjusted values provide for less webbing effect due to motion variations of the catheter.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system to generate an electroanatomical map of a patient's heart, the system comprising:
a catheter including an electrode configured to detect a plurality of physiological signals from within the patient's heart and a location sensor configured to generate a plurality of location signals representative of a location of the catheter within the patient's heart;
a display device; and
a controller configured to:
determine a catheter context characteristic based on the plurality of location signals;
collect the plurality of physiological signals according to a collection parameter, the collection parameter based on the determined catheter context characteristic;
collect a plurality of anatomical location signals corresponding to a measurement location associated with each of the plurality of physiological signals; and
generate, on the display device, a three-dimensional representation of the patient's heart based on the plurality of physiological signals and the plurality of anatomical location signals.

2. The system of claim 1, wherein the catheter context characteristic is associated with a velocity of the catheter determined using the plurality of location signals.

3. The system of claim 2, wherein the collection parameter includes collecting the physiological signals at a first sampling rate associated with a first context characteristic and at a second sampling rate associated with a second context characteristic.

4. The system of claim 3, wherein the controller configured to generate includes the controller configured to employ a process applying a higher degree of interpolation to the physiological signals collected at the first sampling rate and applying a lower degree of interpolation to the physiological signals collected at the second sampling rate.

5. The system of claim 3, wherein the controller configured to generate includes the controller configured to employ a process applying a higher degree of smoothing to the physiological signals collected at the first sampling rate and applying a lower degree of smoothing to the physiological signals collected at the second sampling rate.

6. The system of claim 3, wherein the first sampling rate is applied when the first context characteristic is a velocity of from about 10 to about 50 mm/s and wherein the wherein the second sampling rate is applied when the second context characteristic is a velocity of from about 2 to about 10 mm/s.

7. The system of claim 1, wherein the mapping process includes a plurality of mapping processes, and each mapping process of the plurality of mapping processes includes an associated set of parameters.

8. The system of claim 1, wherein the parameter is associated with a parameter value, and the parameter value for the parameter is adjusted based on the catheter context characteristic.

9. The system of claim 8, wherein the controller is configured to adjust the parameter value via a lookup table in a memory device.

10. The system of claim 8, wherein the controller is configured to adjust the parameter value via a calculation.

11. The system of claim 1, wherein the catheter includes mapping and ablation electrodes.

12. A method for generating an electroanatomical map of a patient's heart, the method comprising:
receiving a plurality of physiological signals from a catheter within the patient's heart and a plurality of location signals representative of a location of the catheter within the patient's heart;
determining a catheter context characteristic based on the plurality of location signals;
collecting the plurality of physiological signals according to a collection parameter, the collection parameter based on the determined catheter context characteristic;
collecting a plurality of anatomical location signals corresponding to a measurement location associated with each of the plurality of physiological signals; and
generating, on the display device, a three-dimensional representation of the patient's heart based on the plurality of physiological signals and the plurality of anatomical location signals.

13. The method of claim 12, wherein the catheter context characteristic is associated with a velocity of the catheter determined using the plurality of location signals.

14. The method of claim 13, wherein the collecting the plurality of physiological signals according to a collection parameter includes collecting the physiological signals at a first sampling rate associated with a first context characteristic and at a second sampling rate associated with a second context characteristic.

15. The method of claim 14, wherein the generating includes applying a higher degree of interpolation to the physiological signals collected at the first sampling rate and applying a lower degree of interpolation to the physiological signals collected at the second sampling rate.

16. The method of claim 13, wherein the generating includes applying a higher degree of smoothing to the physiological signals collected at the first sampling rate and applying a lower degree of smoothing to the physiological signals collected at the second sampling rate.

17. The method of claim 12, wherein the parameter is associated with a parameter value, and further comprising adjusting the parameter value based on the catheter context characteristic.

18. A system to generate an electroanatomical map of a patient's heart, the system comprising:
a catheter including an electrode configured to detect a plurality of physiological signals from within the patient's heart and a location sensor configured to generate a plurality of location signals representative of a location of the catheter within the patient's heart;
a display device; and
a controller configured to:
determine a catheter context characteristic based on the plurality of location signals;
collect the plurality of physiological signals according to a selected value of a collection parameter from a range of values, the selected value based on the determined catheter context characteristic;
collect a plurality of anatomical location signals corresponding to a measurement location associated with each of the plurality of physiological signals; and
generate, on the display device, a three-dimensional representation of the patient's heart based on the plurality of physiological signals and the plurality of anatomical location signals.

19. The system of claim 18, wherein the controller is configured to select the value via a lookup table.

20. The system of claim 19, wherein the controller is configured to input a velocity of the catheter into the lookup table to obtain the selected value.
